(19) United States
(12) Patent Application Publication
(10) Pub. No.: US 2026/0198769 A1
(43) Pub. Date: Jul. 16, 2026

(54) VISION EVALUATION SYSTEM

(71) Applicant: MENICON CO., LTD, Kasugai (JP)

(72) Inventors: Tsuyoshi OKAMOTO, Bedford, MA (US); Stephen D. NEWMAN, Bayshore Park (SG); Bruce MAI, Woodland Rings Road (SG)

(73) Assignee: MENICON CO., LTD, Kasugai (JP)

(21) Appl. No.: 19/016,592

(22) Filed: Jan. 10, 2025

(51) Int. Cl. A61B 3/032; A61B 3/00

(52) U.S. Cl. CPC A61B 3/032; A61B 3/0025

(57) ABSTRACT

A vision evaluation system is provided that includes portable device with a display, and a controller. The vision evaluation system can operate in a mode in which the controller defines a virtual space and displays an image on the display that represents a portion of the virtual space that is based the position of the portable device. The vision evaluation system can display a visual target that moves or relocates once the user tags the visual target by tapping it or by gazing at it.

### BACKGROUND

Vision evaluation systems use software and equipment to train, monitor, measure, or otherwise evaluate a user's vision acuity or eye movement. In existing systems, a display device can display a visual marker or target towards the edges of the display area so that if a user's vision is fixed at the center of the display screen, the target will be in the user's peripheral vision. In this way, the user can identify or touch the markers, and the user's peripheral vision acuity can be assessed. Similarly, to provide eye movement evaluation, a visual target can be displayed at various portions of the display screen with the user's head in a fixed position so that the user's eyes can be trained to follow the visual target on the screen.

However, existing systems are limited by the display size. In this regard, if the display screen is too small, visual targets that are displayed toward the edge of the screen may be located too close to the user's vision center. Alternatively, to overcome this problem, some systems use a very large display so that the visual target at the edge of the display area is sufficiently located in the user's peripheral vision. However, large displays are more expensive and cumbersome.

### SUMMARY

It was discovered in connection with this disclosure that a user's eye-movement or peripheral vision can be effectively evaluated by using a portable or hand-held display to display an image representing a portion of a virtual space, and depicting a visual target in the image at certain locations in the virtual space based, e.g., on the user's gaze and/or facial orientation. Accordingly, in some embodiments, suitable vision evaluation can be provided even if a small display is used since the display can be moved to any region in the virtual space to display the visual target including regions corresponding to a user's periphery.

In accordance with one aspect, this disclosure provides a system for evaluating a user's vision with a portable device that includes a display. The system includes a controller with at least one processor that is programmed to (a) set a reference position of the portable device, (b) determine a relative spatial orientation between the portable device and the reference position, (c) determine a virtual position of the portable device in a virtual space based on the relative spatial orientation, (d) cause an image to be displayed on the display that represents a portion of the virtual space that corresponds to the virtual position of the portable device, (e) determine a position of a visual target in the virtual space and, if the position of the visual target is within the portion of the virtual space, cause the visual target to be displayed as part of the image, and (f) change the position of the visual target in the virtual space to a new position that is outside of the displayed image in response to the user tagging the visual target by either (i) touching the display when the visual target is displayed in the image; or (ii) gazing at the visual target.

In accordance with another aspect, this disclosure provides a system for evaluating a user's vision with a portable display device that includes a camera and a display. The system includes a controller with at least one processor that is programmed to (a) determine the user's gaze based on a camera image of the user taken from the camera; (b) determine an area of the display that is in the user's peripheral vision based on the user's gaze; (c) display an image on the display that includes a visual target in the area; and (d) then determine if the user's gaze is directed at the visual target, and if the user's gaze is directed at the visual target, change the display position of the visual target to a new position on the display.

### BRIEF DESCRIPTION OF THE DRAWINGS

FIG. 1 is a perspective view of portable display device that can be used with embodiments of the vision evaluation system;

FIG. 2 is a schematic illustration of a user operating the vision evaluation system;

FIGS. 3A and 3B are schematic illustrations of screen shots of an electronic device operating in a first mode of the vision evaluation system.

FIGS. 4A and 4B are schematic illustrations of screen shots of an electronic device operating in a second mode of the vision evaluation system.

FIG. 5 is a schematic illustration of a screen shot of an electronic device operating in a third mode of the vision evaluation system.

### DETAILED DESCRIPTION OF EMBODIMENTS

In the following description, numerous details are set forth to provide an understanding of the present disclosure. However, it may be understood by those skilled in the art that the methods and systems of the present disclosure may be practiced without these details and that numerous variations or modifications from the described embodiments may be possible.

Embodiments of the invention provide a vision evaluation system that can assist in assessing a user's visual acuity, making a user's peripheral vision more accurate, improving a user's response time to images in peripheral areas, and improve a user's eye movement and dynamic vision.

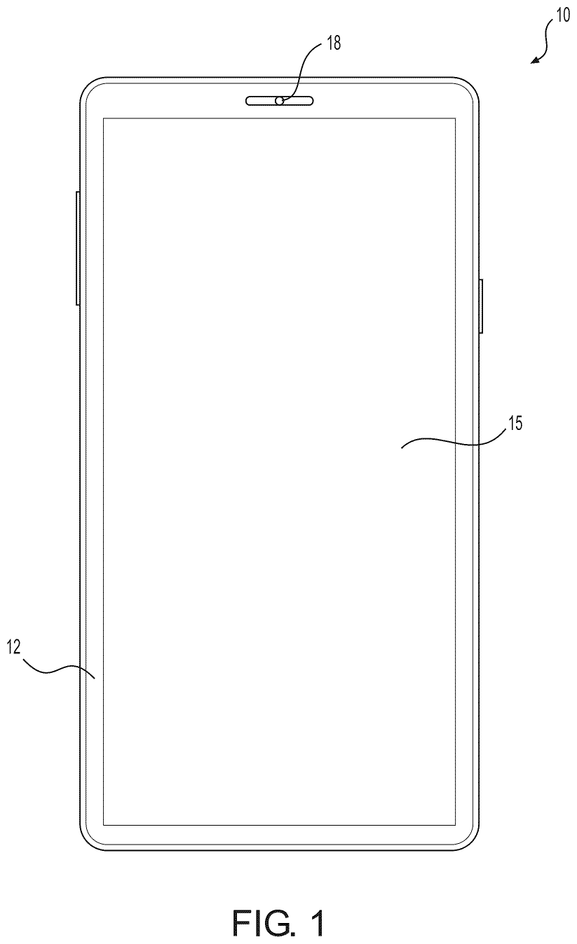

Referring to FIG. 1, the vision evaluation system in one embodiment can be implemented on a portable device 10. The portable device 10 can be a cellular phone such a smartphone, a tablet computer, or a laptop. The portable device 10 is preferably sized to be handheld and having a largest dimension that is less than 50 cm or less than 25 cm, for example.

The portable device 10 includes a housing 12, a display 15, and at least one camera 18. The portable device 10 includes a controller and a memory, which are not shown in FIG. 1. The controller includes at least one processor that can be programmed with software to perform vision evaluation for a user. The controller can render images to implement training vision exercise or program for evaluating the user's vision, where the images are displayed on display 15. In some embodiments, the controller or at least one of the processors of the controller can be present on another device that is remote from the portable device 10, e.g., on a remote server, which can communicate with the portable device over a communications network.

The display 15 can be a touchscreen that can detect a user's touch by using known techniques such as capacitance sensing or light sensing. The at least one camera 18 can include a visible light camera and/or an infrared camera, for example. The camera 18 can face in the same direction as the display 15 and can take images of a user as the user faces the display 15 and interacts with the vision evaluation system. The portable device 10 can also include at least one camera on the opposite side of the portable device 10 that can take images of the user's surroundings while the user is facing the display 15.

The portable device 10 can include one or more motion sensors that measure a motion of the portable device. For example, the portable device can include a gyro sensor that can sense angular rotational velocity and angular acceleration. The portable device 10 can also include one or more accelerometers that can sense acceleration forces in at least one direction, and preferably in two or three dimensions.

The controller can be programmed with software that uses images from the camera 18 to continuously track the user's eyes and head (i.e., at a frequency of over 50 samples per second). The software can track orientation (e.g., rotational position) and movement (e.g., rotational speed) of each of the user's eyes, and can use this information to determine the user's gaze (i.e., where the user is looking). The software can also track the user's head position, head orientation (e.g., rotational position), and head movement rate. Such software is readily available including, for example, ARKit, RealSense, Tobii, etc.

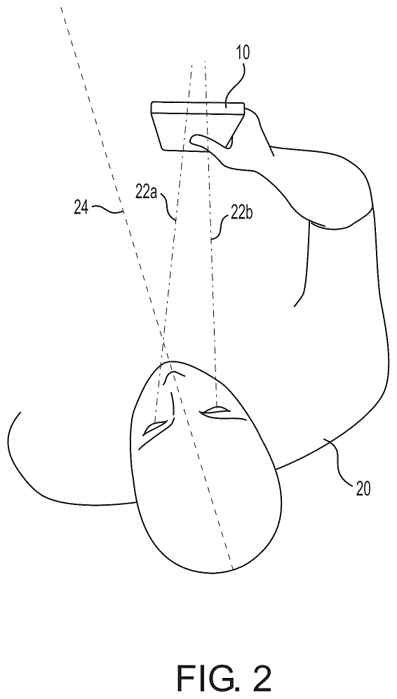

The at least one processor of the portable device 10 can be programmed with vision evaluation software that causes images to be displayed on display 15, as described below in connection with FIGS. 2-4.

In embodiments of the visual evaluation system, e.g., as described in connection with FIGS. 2-4 below, the processor can be programmed to render images corresponding to different portions of a virtual space in either two or three dimensions. The virtual space is larger than the size of display 15 such that display 15 can only depict an image 30 corresponding to a portion of the virtual space at a given time. The user can move portable device 10 in real space so that the portable device displays different portions of the virtual space depending on its position in the virtual space. The system first determines a reference position of the portable device 10, which can be a position of the portable device 10 when the vision exercise is started. The reference position can be assigned to correspond to a position in the virtual space, e.g., such as the center or middle part of the virtual space. As the user moves the portable device 10 in real space, the controller can determine the position of the portable device 10 based on signals received from motion sensors in the device, such as a gyro sensor and an accelerometer. The controller can then determine the relative spatial orientation between the portable device 10 and the reference position, which is used to determine the location of the portable device in the virtual space.

As an alternative or in addition to using motion sensors, the reference position of the portable device can be set relative to the user or a part of the user (e.g., the user's head) based on images taken from the camera 18. The relative spatial orientation between the user and the device 10 can then be evaluated by known techniques such as by using images from camera 18 to determine the size and orientation of a user's head. If the user's head is not within the field of view of camera 18 and the controller cannot determine the location of the portable device 10 in the virtual space, the controller can cause an error message to be displayed on display 15 or an instruction that prompts the user to reposition the portable device 10 so that the camera 18 is facing the user (i.e., “Please turn the screen towards you.”).

FIG. 2 is a schematic illustration of a user 20 operating a vision evaluation system according to embodiments of the invention. The user 20 is holding portable device 10 in his right hand. The portable device 10 can evaluate the user's left eye orientation 22a and right eye orientation 22b to determine the user's gaze. In the illustrated example, the user's gaze coincides with the display of the portable device 10. The portable device 10 can also determine its location in the virtual space, e.g., based on the relative spatial orientation with a reference position, optionally by using the motion sensors described above and tracking how much the device 10 has moved from its previous position. The portable device 10 can also track the user's head/face orientation, e.g., by detecting the face axis 24.

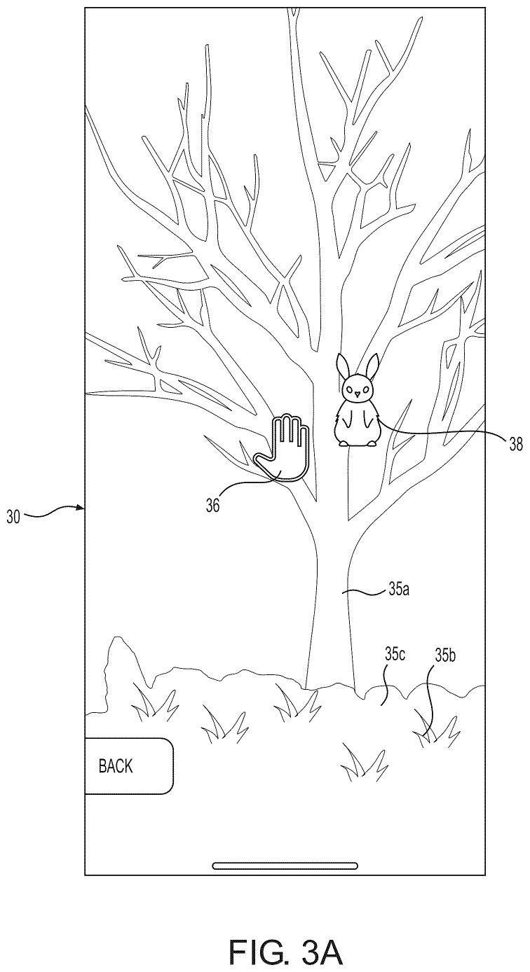

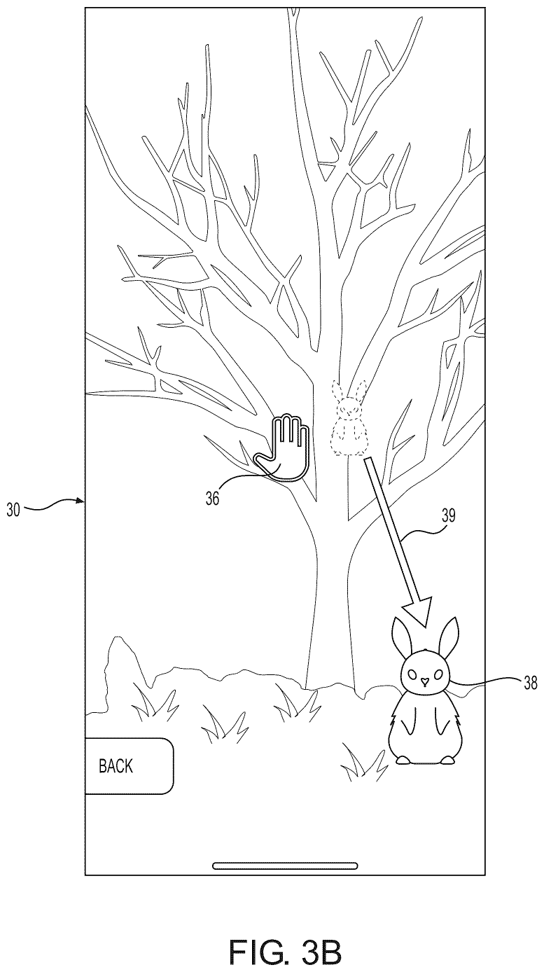

FIGS. 3A and 3B illustrate screenshots of an embodiment of the vision evaluation system with a two-dimensional (2D) virtual space. FIG. 3A shows a display image 30 corresponding to a portion of the 2D virtual space. The image 30 includes objects such as a tree 35a, bush 35b, and ground 35c. In the portion of the visual space represented by image 30, a visual target 38 is displayed, which may be a character, mascot, an object, a graphic symbol, etc. The image 30 also includes an icon 36, which is located on a fixed portion of the image relative to the display (e.g., at the center of the display) so that when the user moves the portable device, the icon 36 remains fixed at the position on the display and appears to move relative to other objects in the image 30.

In this vision evaluation mode, the user can move the portable device 10 around (e.g., side-to-side, up-and-down, forward-and-rearward) with his arm in real space, and the controller can determine the location of the portable device 10 in the virtual space based on motion sensors, as described above. The portable device 10 can display an image 30 on the display that corresponds to the portion of the virtual space at which the device is located. If the image 30 corresponds to the position in the virtual space where the visual target 38 is located, the visual target 38 can also be displayed in the image 30. And, as the user moves the portable device 10 in real space, the image 30 appears to be moving on the display 15 as well.

The processor can be programmed to select the position of the visual target 38 in the virtual space based on the user's head orientation and/or gaze. In this regard, the processor can be programmed to display the visual target 38 in the user's peripheral area relative to the user's face orientation. For example, the processor can select a location of the visual target 38 in the virtual space so that it would have an angle with respect to the face axis (e.g., line 24 in FIG. 2) that is within a range of from 25° to 100°, from 35° to 90°, or from 45° to 80°, for example. The processor can also be programmed to determine the position of the visual target based on predetermined settings in the vision evaluation system. For example, the system settings that can affect the location of the visual target 38 include the size of the virtual space, the speed of the movement of the visual target 38, level of difficulty in finding the visual target 38, etc. In some aspects, the controller can also randomly determine the position of the visual target 38 in the virtual space.

As shown in FIG. 3A, the icon 36 is at a fixed position on the display 15, such as in the center of the display. In this embodiment, the user tags the visual target 38 by positioning the icon 36 over the visual target 38 so that the visual target 38 is at the known or predefined position on the display, and tapping or otherwise touching the display (at any location on the display). Tagging the visual target 38 in this way causes the at least one processor to change positions of the visual target 38 in the virtual space. The icon 36 can be animated when the user touches the display (e.g., by showing a grabbing motion, changing color, etc.). As an alternative to using the icon 36 to tag the visual target 38, the user can tag the visual target 38 by tapping or otherwise touching the display at a position corresponding to the visual target 38 (e.g., the user touches the display on the visual target 38 or in close proximity to the visual target 38), or the user can tag the visual target 38 by tapping or otherwise touching any portion of the display when the visual target appears in image 30. In other embodiments, the user can tag the visual target 38 by gazing at it. In such a case, the processor can evaluate the user's gaze based on images from camera 18 and can determine when the user's gaze coincides with the visual target 38.

The portable device 10 can also change positions of the visual target 38 in the virtual space if the user does not tag or gaze at the visual target 38 within a certain time period, e.g., after the visual target 38 changes to its new position in the virtual space or after the visual target 38 appears on the display 15.

FIG. 3B illustrates the image 30 of the virtual space once the position of the visual target 38 is changed in response to being tagged. Once the visual target 38 is tagged as described above or the allotted time expires, the processor can determine a new position of the visual target 38 in the virtual space, which is usually in a portion of the virtual space that is not displayed in the current image 30. In FIG. 3B, the visual target 38 is shown moving toward its new position off screen in direction of arrow 39. Arrow 39 is not displayed in this embodiment, and instead represents displayed sequential movement of the visual target 38 across the display as the visual target 38 moves out of the image 30 (e.g., the visual target 38 can be displayed as running or walking off-screen). Once the visual target 38 is tagged, the speed and direction of movement of the visual target 38 can be determined based on its new position in the virtual space. Accordingly, the speed and direction of movement can notify the user of the new position of the visual target 38 so that the user can seek out the new position of the visual target 38. For example, if the new position is located far away in the virtual space, the visual target 38 can move off-screen relatively quickly, and if the new position is located relatively near in the virtual space, the visual target 38 can move off-screen relatively slowly.

As an alternative to showing the visual target 38 move off-screen, the visual target 38 can instead vanish from its current position once it is tagged, or once the allotted time expires. The visual target 38 can be relocated at the new position in the virtual space that is off-screen from the current image 30. In such a case, the processor can display arrows or other graphical indicators on the image 30, or provide audio prompts, that point toward the new position of the visual target 38 in the virtual space.

In some embodiments, the new position of the visual target 38 and/or the speed in which it moves off-screen once tagged by the user can depend on how quickly and/or accurately the user tags the visual target 38. In this regard, if the user is able to tag the visual target 38 quickly, the controller can make it more difficult for the user to find the visual target 38 at the next position by moving the visual target 38 far away in the virtual space (in 3D space the distance from the user along particular axes may also make the visual target appear smaller) and/or by moving the visual target 38 off-screen from current image 30 quickly. Conversely, if the user is slow to tag the visual target 38, the controller can make it easier for the user to find the visual target 38 at the next position by moving the visual target 38 to a relatively nearer position in the virtual space and/or by moving the visual target 38 off-screen from the current image 30 relatively slowly. The controller can assess the user's speed in tagging the visual target 38 based on the duration from when the visual target 38 appears at its position in the virtual space to the time the user tags the visual target 38, or possibly based on the duration from when the visual target 38 appears on the display image from the time the user tags the visual target 38. The controller can also assess the user's accuracy in tagging the visual target 38 by determining the distance from the user's contact on the display 15 to the location of the visual target on the display 15. For example, the controller can assign zero points where it detects that a user has tapped the display and the visual target 38 is not displayed in image 30. The controller can assign five points where the location of the user's tap does not overlap or correspond to the location of the visual target 38 but the entire visual target 38 is nonetheless displayed on image 30. The controller can assign ten points where user's tap touches at least part of the visual target 38.

The controller can also control other variables to train the user's vision in different ways or to make the vision exercise more or less challenging for the user, or to improve eye movement and dynamic vision. As above, these variables can be controlled based on the user's performance to make the exercise more challenging when the user is quickly tagging the visual target 38 and to make the exercise less challenging when the user is more slowly tagging the visual target 38. As one example, the controller can adjust the brightness of the display to control the contrast sensitivity. The backlight can be made relatively dimmer to make the exercise more challenging for the user or can be made brighter to make the exercise less challenging for the user. The brightness can also be controlled based on detected ambient light. As another example, the controller can change the color of the visual target 38 and/or the background of the image 30 so that there is less color contrast or more color contrast. Similarly, the controller can control the position of the visual target 38 so that it is superimposed on portions of the image 30 that have similar colors to reduce the color contrast, or so that it is superimposed on dissimilar colors to increase the color contrast. As another example, the controller can control the size of the visual target 38 to make the visual target 38 smaller or larger. The controller can also position and display the visual target 38 on the image 30 so that it appears hidden or partially hidden behind objects. For example, referring to FIG. 3A, the visual target 38 can be displayed to be partially behind tree 35a to make the exercise more challenging, or can be displayed to be completely hidden by objects so that the user has to guess where the visual target is located.

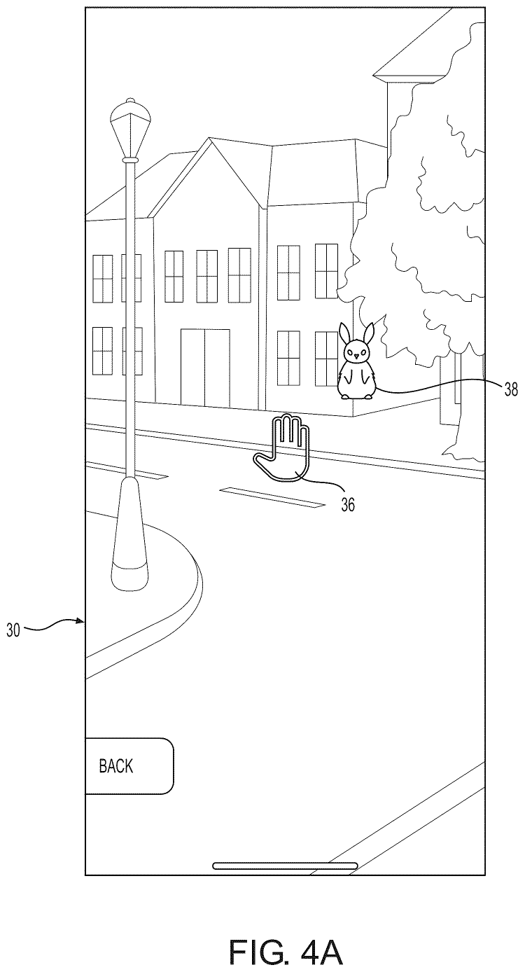

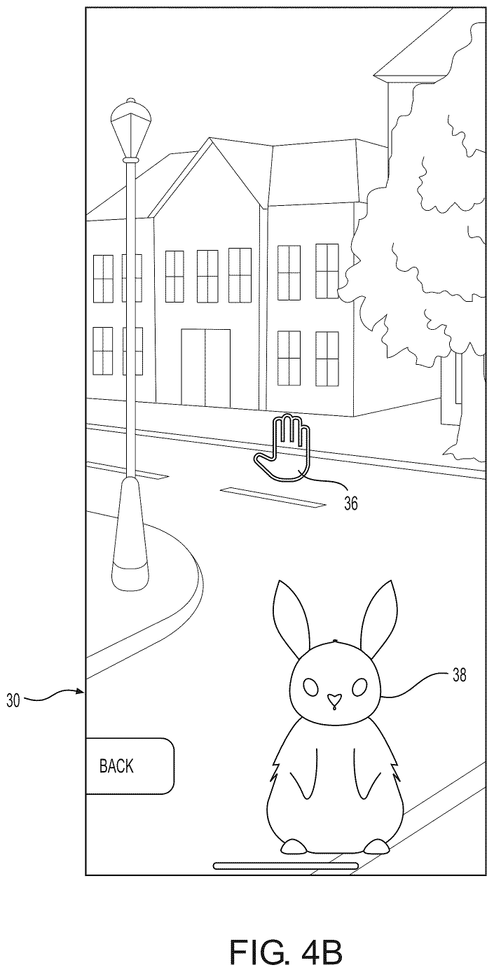

FIGS. 4A and 4B illustrate screenshots of an embodiment of the vision evaluation system with a three-dimensional (3D) virtual space. The features of the vision evaluation system in this mode can be the same as in the 2D mode, as described above, with the exception that the virtual space is defined three-dimensionally and thus the visual target 38 can be positioned anywhere in the 3D space and the controller must determine where the portable device 10 is in the 3D space to determine which portion of the 3D virtual space to display on the image 30. The controller can determine the reference position of the portable device 10, as well as the relative spatial orientation of the portable device 10 to the reference position in the same manner as described above in connection with the other embodiments. In this case, as the user moves the device in real space, the virtual space objects can be seen moving in the image 30 in three dimensions. For example, if the visual target 38 is displayed in image 30 and the user moves the device toward the visual target 38 in the forward direction (or z-axis direction), the visual target 38 will appear to get larger, and if the user moves the device away from the visual target in the rearward direction, the visual target 38 will appear to get smaller. Once the user tags the visual target 38, the controller can also move the visual target 38 off-screen to a new position. In this embodiment, since the space is three-dimensional, the visual target 38 can be depicted as moving in the 3D space. For example, FIG. 4B illustrates the relative movement of the visual target 38 from FIG. 4A after the user tags the visual target 38 where the visual target 38. As shown, the visual target 38 is depicted as getting larger as it comes toward the user and off-screen (e.g., to a new position that is located behind the user in the virtual space).

In the embodiments described above, the virtual space, and the display images, can be depicted as a virtual reality (VR) space where the background and objects are created by computer graphics, or alternatively can be generated as an augmented reality (AR) space where the space is depicted as the user's environment in real time and the visual target 38 is superimposed or overlaid over images of the user's environment.

The controller can track and assess the user's performance in the vision evaluation system. The controller can track the user's tagging accuracy and tagging speed under the different conditions described above, which can be weighted based on difficulty level, and can assign the user an overall score based on this information that can be displayed. This helps to motivate the user and encourages the user to continue using the vision evaluation system. The controller can also determine fast and slow reaction points in the field of view of the user based on the user's face orientation and position of the visual target when the screen is tapped. Based on this information, the controller can create and display a map of positions in the user's field of view where the user responds quickly and where the user responds more slowly.

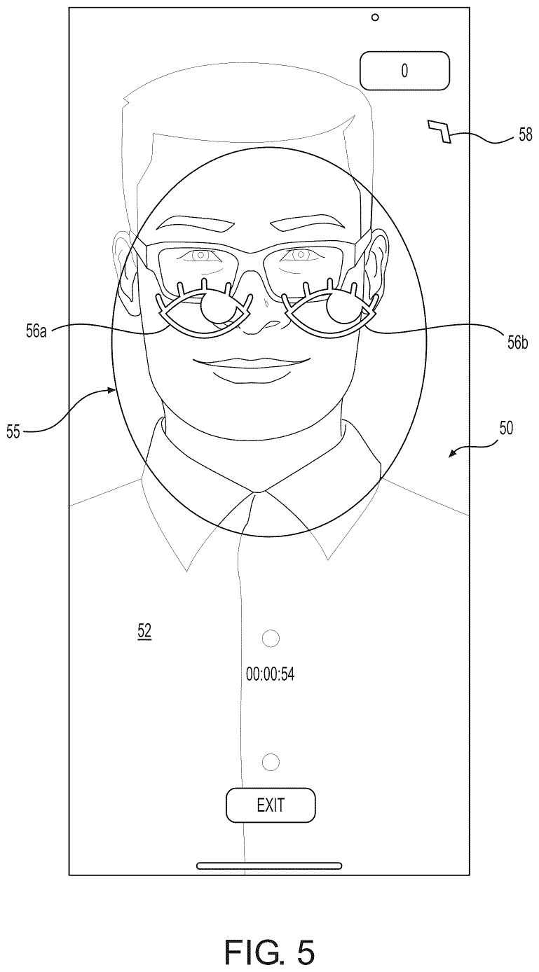

FIG. 5 is a schematic illustration of a screen shot of an electronic device operating the vision evaluation system in a third mode, which is an eye exercise mode. In this mode, camera 18 of the portable device 10 faces the user and takes images of the user 52 as described above, and software on the portable device 10 tracks the user's gaze based on detected eye rotation. In this mode, the user can maintain their head orientation in a fixed position and moves their eyes in response to visual or audio prompts. The portable device 10 can display an image 50 of the user 52 on the display 15. The image 50 can also include a visual guide 55 for positioning the user's head relative to the display 15. The visual guide 55 can include a shape such as an oval that shows where the user's head is positioned. The portable device 10 can instruct the user to maintain their head in a fixed position, and can then display visual targets 58 on image 50 that the user follows with their eyes.

The processor can select a location of the visual target 58 on image 50 so that the visual target 58 would have an angle with respect to the face axis (e.g., line 24 in FIG. 2) that is within a range of from 25° to 100°, from 35° to 90°, or from 45° to 80°, for example. The visual target 58 can move continuously on image 50 so that the user follows the visual target 58 around, or the visual target 58 can vanish and reappear at a new location on the image 50. The portable device 10 can determine when the user's gaze coincides with the visual target 58 and can reposition or move the visual target 58 once the user's gaze falls upon the visual target 58. The speed at which the visual target 58 moves can be set in advance, or the controller can change the speed of the visual target 58 in response to the user's performance, e.g., to increase the speed if the user is performing well and vice-versa.

In some embodiments, the processor can also display eye guides 56a, 56b that illustrate where the user's gaze should be directed so that the user's gaze can be fixed, and then display the visual target 58 in the user's peripheral vision area, e.g., from 35° to 90°, or from 45° to 80°, from a central axis of the user's gaze.

As above, the controller can similarly track the user's performance in the eye exercise mode and can display a score that corresponds to the user's performance or provides a report relating to the user's performance.

It will be appreciated that the above-disclosed features and functions, or alternatives thereof, may be desirably combined into different methods and systems. Also, various alternatives, modifications, variations or improvements may be subsequently made by those skilled in the art, and are also intended to be encompassed by the disclosed embodiments. As such, various changes may be made without departing from the spirit and scope of this disclosure.

What is claimed is

1. A system for evaluating a user's vision with a portable device that includes a display, the system comprising:
a controller with at least one processor that is programmed to:
(a) set a reference position of the portable device;
(b) determine a relative spatial orientation between the portable device and the reference position;
(c) determine a virtual position of the portable device in a virtual space based on the relative spatial orientation;
(d) cause an image to be displayed on the display that represents a portion of the virtual space that corresponds to the virtual position of the portable device;
(e) determine a position of a visual target in the virtual space and, if the position of the visual target is within the portion of the virtual space, cause the visual target to be displayed as part of the image; and
(f) change the position of the visual target in the virtual space to a new position that is outside of the displayed image in response to the user tagging the visual target by either (i) touching the display when the visual target is displayed in the image; or (ii) gazing at the visual target.

2. The system of claim 1, wherein the portable device includes at least one motion sensor, and wherein the at least one processor is programmed to determine a position of the portable device based on signals from the at least one motion sensor and to determine the relative spatial orientation based on the position of the portable device.

3. The system of claim 2, wherein the at least one motion sensor includes a gyro sensor and an accelerometer.

4. The system of claim 1, wherein the portable device includes a camera, and the at least one processor is programmed to determine a facial orientation of the user based on a camera image of the user taken from the camera.

5. The system of claim 4, wherein the at least one processor is programmed to cause the visual target to be displayed in the image at a position that is based on the determined facial orientation of the user.

6. The system of claim 1, wherein the at least one processor is programmed to display the visual target moving out of the image to the new position in response to the user tagging the visual target.

7. The system of claim 1, wherein the at least one processor is programmed to change the position of the visual target in the virtual space to the new position in response to the user tagging the visual target by: touching a position on the display corresponding to the visual target, touching the display when the visual target is displayed anywhere on the display, or touching the display when the visual target is at a predefined position on the display.

8. The system of claim 6, wherein the at least one processor is programmed to control a speed and direction at which the visual target moves out of the image based on the new position of the visual target in the virtual space.

9. The system of claim 8, wherein the at least one processor is programmed to cause the visual target to move out of the image relatively quickly when the new position is relatively far in the virtual space from the position of the visual target, and cause the visual target to move out of the image relatively slowly when the new position is relatively near in the virtual space from the position of the visual target.

10. The system of claim 1, wherein the at least one processor is programmed to track at least one of the user's speed and the user's accuracy in tagging the visual target.

11. The system of claim 10, wherein the at least one processor is programmed to assign a score based on the at least one of the user's speed and the user's accuracy, and to display the assigned score on the display.

12. The system of claim 6, wherein the at least one processor is programmed to control a speed at which the visual target moves out of the image based on the user's speed in tagging the visual target.

13. The system of claim 6, wherein the at least one processor is programmed to control a speed at which the visual target moves out of the image based on predetermined settings.

14. The system of claim 1, wherein the at least one processor is programmed to determine the new position of the visual target in the virtual space based on the user's speed in tagging the visual target.

15. The system of claim 1, wherein the at least one processor is programmed to render images corresponding to different portions of the virtual space, and wherein the virtual space is larger than the display.

16. The system of claim 1, wherein the at least one processor is programmed to render images corresponding to different portions of the virtual space, and the virtual space is either a virtual reality (VR) space in which the images are computer graphics, or is an augmented reality (AR) space in which the images show the user's real world environment.

17. The system of claim 1, wherein the at least one processor is programmed to control a brightness of the display based on at least one of the user's speed and the user's accuracy in tagging the visual target.

18. The system of claim 1, wherein the at least one processor is programmed to control a color contrast between the visual target and the image based on the at least one of the user's speed and the user's accuracy in tagging the visual target.

19. A system for evaluating a user's vision with a portable display device that includes a camera and a display, the system comprising:
a controller with at least one processor that is programmed to:
(a) determine the user's gaze based on a camera image of the user taken from the camera;
(b) determine an area of the display that is in the user's peripheral vision based on the user's gaze;
(c) display an image on the display that includes a visual target in the area; and
(d) then determine if the user's gaze is directed at the visual target, and if the user's gaze is directed at the visual target, change the display position of the visual target to a new position on the display.

20. The system of claim 19, wherein the at least one processor is programmed to display a visual guide on the image for positioning the user's head relative to the display.
